(19) United States
(12) Patent Application Publication
(10) Pub. No.: US 2021/0393715 A1
(43) Pub. Date: Dec. 23, 2021

(54) ANTI-CANCER-ASSOCIATED NON-TUMOR CELL AGENT COMPRISING VIRUS

(71) Applicants: NATIONAL UNIVERSITY CORPORATION OKAYAMA UNIVERSITY, Okayama-shi (JP); Oncolys BioPharma Inc., Tokyo (JP)

(72) Inventors: Toshiyoshi FUJIWARA, Okayama (JP); Toshihiro OGAWA, Okayama (JP); Takeyoshi NISHIYAMA, Okayama (JP); Satoru KIKUCHI, Okayama (JP); Hiroshi TAZAWA, Okayama (JP)

(73) Assignees: NATIONAL UNIVERSITY CORPORATION OKAYAMA UNIVERSITY, Okayama-shi (JP); Oncolys BioPharma Inc., Tokyo (JP)

(21) Appl. No.: 17/352,576

(22) Filed: Jun. 21, 2021

(30) Foreign Application Priority Data
Jun. 19, 2020 (JP) 2020-106251

(51) Int. Cl. A61K 35/768; A61P 35/00; A61K 35/33

(52) U.S. Cl. CPC A61K 35/768; A61K 35/33; A61P 35/00

(57) ABSTRACT

In one embodiment, the present invention provides an anti-cancer-associated non-tumor cell agent.

In one embodiment, the present invention relates to an anti-cancer-associated non-tumor cell agent comprising an oncolytic virus comprising a p53 gene. In one embodiment, the present invention relates to an anti-cancer-associated non-tumor cell preparation comprising a recombinant virus comprising: a first gene cassette containing a telomerase reverse transcriptase promoter, an E1A gene, an IRES sequence and an E1B gene; and a second gene cassette containing a promoter and a p53 gene.

### FIELD OF THE INVENTION

The present invention relates to an anti-cancer-associated non-tumor cell agent comprising a virus, and a pharmaceutical composition comprising the anti-cancer-associated non-tumor cell agent.

### BACKGROUND OF THE INVENTION

Tumor microenvironments are considered to play an important role in growth, progression, infiltration, metastasis, angiogenesis, metabolism, immunosuppression, chemotherapy resistance and the like of tumors. The tumor microenvironment is constituted by cancer-associated non-tumor cells, extracellular matrices and the like, and the cancer-associated non-tumor cells include tumor-associated fibroblasts (cancer-associated fibroblasts, CAFs), tumor-associated macrophages, immune inflammatory cells, and mesenchymal stem cells.

Since the tumor microenvironment is involved in growth of the tumor, etc. as described above, it is considered that a drug which damages cancer-associated non-tumor cells constituting the tumor microenvironment can be used for treatment and/or prevention of cancer. In addition, since the tumor microenvironment is also involved in drug resistance, it is considered that a drug which damages cancer-associated non-tumor cells can be used for enhancing the effect of anticancer agents in cancers having drug resistance, etc.

However, an effective anti-cancer-associated non-tumor cell agent has not been heretofore known.

In one embodiment, an object of the present invention is to provide an anti-cancer-associated non-tumor cell agent.

### BRIEF SUMMARY OF THE INVENTION

The present inventors have found that an oncolytic virus comprising a p53 gene, and a recombinant virus comprising a first gene cassette containing a telomerase reverse transcriptase promoter, an E1A gene, an IRES sequence and an E1B gene and a second gene cassette containing a promoter and a p53 gene damages cancer-associated non-tumor cells, leading to completion of the present invention.

The present invention encompasses the following embodiments.

(1) An anti-cancer-associated non-tumor cell agent comprising an oncolytic virus comprising a p53 gene.
(2) An anti-cancer-associated non-tumor cell agent comprising a recombinant virus comprising:

a first gene cassette containing a telomerase reverse transcriptase promoter, an E1A gene, an IRES sequence and an E1B gene; and

a second gene cassette containing a promoter and a p53 gene.

(3) The anti-cancer-associated non-tumor cell agent according to (2), wherein the recombinant virus is an oncolytic virus.
(4) The anti-cancer-associated non-tumor cell agent according to (2) or (3), wherein the first gene cassette contains a telomerase reverse transcriptase promoter, an E1A gene, an IRES sequence and an E1B gene in this order.
(5) The anti-cancer-associated non-tumor cell agent according to any one of (2) to (4), wherein the promoter in the second gene cassette is an Egr1 promoter.
(6) The anti-cancer-associated non-tumor cell agent according to any one of (1) to (5), wherein the cancer-associated non-tumor cell is a cancer-associated fibroblast.
(7) The anti-cancer-associated non-tumor cell agent according to any one of (1) to (6), wherein the virus is a recombinant adenovirus.
(8) The anti-cancer-associated non-tumor cell agent according to any one of (1) to (7), wherein the virus treats and/or prevents a cancer by damaging cancer-associated non-tumor cells.
(9) The anti-cancer-associated non-tumor cell agent according to (8), wherein the cancer is pancreas cancer or stomach cancer.
(10) A pharmaceutical composition comprising the anti-cancer-associated non-tumor cell agent according to any one of (1) to (9).
(11) A method for damaging a cancer-associated non-tumor cell, comprising administering an oncolytic virus comprising a p53 gene to a subject.
(12) A method for damaging a cancer-associated non-tumor cell, comprising administering, to a subject, a recombinant virus comprising:

a first gene cassette containing a telomerase reverse transcriptase promoter, an E1A gene, an IRES sequence and an E1B gene; and

a second gene cassette containing a promoter and a p53 gene.

(13) The method according to (12), wherein the recombinant virus is an oncolytic virus.
(14) The method according to (12) or (13), wherein the first gene cassette contains a telomerase reverse transcriptase promoter, an E1A gene, an IRES sequence and an E1B gene in this order.
(15) The method according to any one of (12) to (14), wherein the promoter in the second gene cassette is an Egr1 promoter.
(16) The method according to any one of (11) to (15), wherein the cancer-associated non-tumor cell is a cancer-associated fibroblast.
(17) The method according to any one of (11) to (16), wherein the virus is a recombinant adenovirus.
(18) The method according to any one of (11) to (17), wherein the virus treats and/or prevents a cancer by damaging cancer-associated non-tumor cells.
(19) The method according to (18), wherein the cancer is pancreas cancer or stomach cancer.
(20) The method according to any one of (11) to (19), wherein the virus is administered as a pharmaceutical composition.

The present application claims the priority to Japanese Patent Application No. 2020-106251, the disclosure of which is herein incorporated by reference.

### BRIEF DESCRIPTION OF THE DRAWINGS

The patent or application file contains at least one color drawing. Copies of this patent or patent application publication with color drawing will be provided by the USPTO upon request and payment of the necessary fee.

FIGS. 1A to 1C are schematic diagrams of a recombinant adenovirus used in Examples. FIG. 1A is a schematic diagram of OBP-702. OBP-702 lacks an E1 gene, and contains, at the position thereof, a gene cassette containing hTERT-p, E1A, IRES and E1B. OBP-702 lacks an E3 gene, and contains, at the position thereof, a gene cassette containing a mouse Egr1 promoter (Egr1-p) and p53. FIG. 1B is a schematic diagram of OBP-301. OBP-301 lacks an E1 gene, and contains, at the position thereof, a gene cassette containing hTERT-p, E1A, IRES and E1B. FIG. 1C is a schematic diagram of Ad-p53. Ad-p53 lacks E1 and E3 genes, and contains, at the position of the E1 gene, a gene cassette containing CMV-p and p53.

FIGS. 2A and 2B show the results of western blotting (A) and the result of immunostaining (B) in a CAF model prepared by treating fibroblasts (FEF3 or GF) with a cancer conditioned medium (CM) or TGF-β.

FIGS. 3A and 3B show the cell viability after treatment of normal gastric fibroblasts (GFs) and CAF model cells (TGF-β-activated GFs) with OBP-702 (A) and paclitaxel (PTX) (B).

FIGS. 4A and 4B show the results of measuring the effects of OBP-702 on expression of various proteins (A) and production of IL-6 (B) in CAF model cells by western blotting (A) and ELISA (B).

FIGS. 5A and 5B show the cell viability after treatment of a CAF clinical sample (A) and normal fibroblasts (NFs) (B) with OPB-702, OPB-301, PTX and Ad-p53.

FIG. 6A shows the results of a cell proliferation assay of a CAF model obtained by treating pancreatic stellate cells (hPSC-5 or hPSC-14) with a cancer conditioned medium prepared from pancreas cancer cells (Panc-1, MIAPaCa-2, BxPC-3 or Capan-1). FIG. 6B shows the cell viability after a CAF model obtained by treating pancreatic stellate cells with a cancer conditioned medium prepared from Panc-1, MIAPaCa-2, BxPC-3 or Capan-1 is treated with OBP-301 or OBP-702.

FIG. 7 shows the results of western blotting to detect a change in expression of each of various proteins when administering OBP-702 to a CAF model prepared by treating pancreatic stellate cells with a cancer conditioned medium of Panc-1.

FIG. 8A shows the results of a cell growth assay of a CAF model obtained by treating pancreatic stellate cells with TGF-β. FIG. 8B shows the cell viability after a CAF model obtained by treating pancreatic stellate cells with TGF-β is treated with OBP-301 or OBP-702.

FIG. 9A shows the cell viability after a CAF model obtained by treating pancreatic stellate cells (hPSC-5 or hPSC-14) with a cancer conditioned medium prepared from pancreas cancer cells (Panc-1, MIAPaCa-2, BxPC-3 or Capan-1) is treated with AdDL312 or Ad CMVp53. FIG. 9B shows the cell viability after a CAF model obtained by treating pancreatic stellate cells with TGF-β is treated with AdDL312 or Ad CMVp53.

FIG. 10 shows the results of western blotting to detect a change in expression of each of various proteins when administering OBP-702 to a CAF model obtained by treating pancreatic stellate cells (hPSC-5 or hPSC-14) with TGF-β.

FIGS. 11A and 11B show the results of western blotting (A) and the cell viability (B) in a CAF model prepared by treating pancreatic stellate cells (hPSC-5 or hPSC-14) with a cancer conditioned medium of Panc-1 containing a TGF-β inhibitor.

FIGS. 12A and 12B show the results of observing p53 expression (A) and the result of evaluating a change of the tumor nest in terms of major diameter (B) after administration of a virus in a 3D culture tissue.

FIGS. 13A and 13B show the results of observing TUNEL expression in a 3D culture tissue (A) and the result of quantitative determination of the TUNEL expression (B).

FIGS. 14A to 14C show the results of comparison of the tumor volume (A), photographs of the tumors and the result of measuring the tumor weights (B) and changes of the tumors in terms of fold change (C) in preparation of a BxPC-3 mono-injection and BxPC-3+hPSC-14 co-injection subcutaneous tumor model.

FIGS. 15A and 15B show the results of comparison of tumor growth (A) and photographs of the tumors and the result of measuring the tumor weights (B) when administering Mock, OBP-301 and OBP-702 to a BxPC-3+hPSC-14 co-injection model.

FIGS. 16A and 16B show the results of observing p53 expression (A) and the quantitative determination of the p53 expression (B) when administering Mock, OBP-301 and OBP-702 to the BxPC-3+hPSC-14 co-injection model.

### DETAILED DESCRIPTION OF THE INVENTION

In one embodiment, the present invention relates to an anti-cancer-associated non-tumor cell agent comprising or consisting of an oncolytic virus (e.g. recombinant virus) containing a p53 gene (and optionally a promoter described herein).

In one embodiment, the present invention relates to an anti-cancer-associated non-tumor cell agent comprising or consisting of a recombinant virus comprising a first gene cassette and a second gene cassette.

Hereinafter, the oncolytic virus comprising a p53 gene and the recombinant virus comprising a first gene cassette and a second gene cassette are each also referred to simply as a “virus described herein”.

### <1. First Gene Cassette>

In one embodiment, the virus described herein contains a first gene cassette in addition to a second gene cassette described below or in isolation from the second gene cassette. Even when the virus does not contain the second gene cassette, the gene cassette is described as a “first gene cassette” for convenience. The first gene cassette contains a telomerase reverse transcriptase promoter, an E1A gene, an IRES sequence and an E1B gene.

In the recombinant virus described herein, in one embodiment, the E1A gene, the IRES sequence and the E1B gene can be driven by the promoter of the telomerase reverse transcriptase. In tumor cells, the promoter of the telomerase reverse transcriptase is activated, and consequently, the recombinant virus described herein can proliferate. As a result, in the tumor cells, cytotoxicity occurs due to the proliferation of the virus, and consequently, the recombinant virus having the promoter of the telomerase reverse transcriptase can specifically kill tumor cells.

As the telomerase reverse transcriptase (TERT) promoter, one derived from mammals can be used. For example, a TERT promoter derived from humans, mice, rats, cattle or the like can be used. The TERT promoter of mammals including humans is cloned, and the sequence information thereof can be obtained from a database such as NCBI or GenBank. The TERT promoter is preferably a human telomerase reverse transcriptase promoter (hTERT promoter).

The hTERT has been confirmed to have many transcription factor binding sequences in a 1.4 kbp region upstream from the 5′-terminal thereof, and this region is considered to be an hTERT promoter. In particular, the sequence of 181 bp upstream from the translation initiation site is a core region important for expression of the downstream gene. As the hTERT promoter, a sequence including the core region can be used, and for example, a sequence of about 378 bp upstream completely including this core region can be used as the hTERT promoter. The sequence of about 378 bp has been confirmed to be comparable in gene expression efficiency to the core region of 181 bp alone. The nucleotide sequence of an hTERT promoter with a length of 455 bp is set forth as SEQ ID NO: 1. The nucleotide sequence of the core region of 181 bp is set forth as SEQ ID NO: 5.

As the TERT promoter, not only a nucleotide containing a nucleotide sequence set forth as SEQ ID NO: 1 or 5, but also the following nucleotides can be used: (a) a nucleotide which is hybridized under stringent conditions with a nucleotide containing a nucleotide sequence complementary to the nucleotide containing a nucleotide sequence set forth as SEQ ID NO: 1 or 5; (b) a nucleotide containing a nucleotide sequence having a sequence identity of 80% or more, 85% or more, 90% or more, 95% or more, 98% or more or 99% or more with the nucleotide sequence set forth as SEQ ID NO: 1 or 5; and (c) a nucleotide containing a nucleotide sequence having addition, deletion or substitution of one or several bases in the nucleotide sequence set forth as SEQ ID NO: 1 or 5. Preferably, the nucleotides of (a) to (c) have TERT promoter activity.

Examples of the “stringent conditions” herein include conditions of 1×SSC to 2×SSC, 0.1% to 0.5% SDS and 42° C. to 68° C., more specifically conditions in which prehybridization is performed in 1×SSC to 2×SSC with 0.1% to 0.5% SDS at 60 to 68° C. for no less than 30 minutes, followed by performing washing four to six times with 2×SSC with 0.1% SDS at room temperature for 5 to 15 minutes. The detailed procedure of the hybridization method is known, and for example, a reference can be made to “Molecular Cloning, A Laboratory Manual 4th ed.” (Cold Spring Harbor Laboratory (2012)) etc.

The term “sequence identity” of nucleotides herein refers to a proportion (%) of bases identical between two sequences when the two sequences are aligned, and gaps are introduced if necessary to obtain the highest match between the sequences.

The term “several” herein means, for example, two to ten, two to seven, two to five, two to three or two.

The E1A gene and the E1B gene are genes contained in the E1 gene of the adenovirus. The E1 gene means one of early (E) genes among early genes and late (L) genes related to replication of DNA possessed by the virus, and encodes proteins involved in control of transcription of virus genomes. The E1A protein encoded by the E1A gene activates transcription of a group of genes (e.g. E1B, E2 and E4) necessary for production of viruses capable of infection. The E1B protein encoded by the E1B gene helps accumulation of mRNA of the late gene (L gene) in the cytoplasm of infected host cells, and inhibits synthesis of protein in the host cells to promote replication of viruses. The nucleotide sequences of the E1A gene and the E1B gene are set forth as SEQ ID NOS: 2 and 3, respectively. The E1A gene or the E1B gene includes not only a nucleotide containing a nucleotide sequence set forth as SEQ ID NO: 2 or 3, but also (a) a nucleotide which is hybridized under stringent conditions with a nucleotide containing a nucleotide sequence complementary to the nucleotide containing a nucleotide sequence set forth as SEQ ID NO: 2 or 3, (b) a nucleotide containing a nucleotide sequence having a sequence identity of 80% or more, 85% or more, 90% or more, 95% or more, 98% or more or 99% or more with the nucleotide sequence set forth as SEQ ID NO: 2 or 3 and (c) a nucleotide containing a nucleotide sequence having addition, deletion or substitution of one or several bases in the nucleotide sequence set forth as SEQ ID NO: 2 or 3. Preferably, the nucleotides of (a) to (c) encode proteins having E1A activity or E1B activity.

IRES (International Ribosome Entry Site) is a protein synthesis initiation signal specific to Picornaviridae, and is considered to play a role as a ribosome binding site because it has a sequence complementary to the 3′-terminal of 18S ribosome RNA. It is known that the virus-derived mRNA of Picornaviridae is translated through this sequence. The translation efficiency through the IRES sequence is high, so that protein synthesis is performed even from the middle of mRNA, independently from a cap structure. In the virus described herein, both the E1A gene and the E1B gene are independently translated by the promoter of human telomerase. When IRES is used, control of expression by the TERT promoter has an effect independently on the E1A gene and the E1B gene, so that proliferation of viruses can be more strictly limited to cells having telomerase activity as compared to a case where either one of the E1A gene and the E1B gene is controlled by the TERT promoter. The IRES sequence is set forth as SEQ ID NO: 4. The IRES sequence includes not only a nucleotide containing a nucleotide sequence set forth as SEQ ID NO: 4, but also (a) a nucleotide which is hybridized under stringent conditions with a nucleotide containing a nucleotide sequence complementary to the nucleotide containing a nucleotide sequence set forth as SEQ ID NO: 4, (b) a nucleotide containing a nucleotide sequence having a sequence identity of 80% or more, 85% or more, 90% or more, 95% or more, 98% or more or 99% or more with the nucleotide sequence set forth as SEQ ID NO: 4 and (c) a nucleotide containing a nucleotide sequence having addition, deletion or substitution of one or several bases in the nucleotide sequence set forth as SEQ ID NO: 4. Preferably, the nucleotides of (a) to (c) have IRES activity.

The order of the telomerase reverse transcriptase promoter, the E1A gene, the IRES sequence and the E1B gene is not limited as long as the E1A gene and the E1B gene are expressed under control of the telomerase reverse transcriptase promoter. For example, the virus described herein may contain the telomerase reverse transcriptase promoter, the E1A gene, the IRES sequence and the E1B gene in this order from the 5′-side to the 3′-side, or may contain the telomerase reverse transcriptase promoter, the E1B gene, the IRES sequence and the E1A gene in this order from the 5′-side to the 3′-side.

### <2. Second Gene Cassette>

In one embodiment, the virus described herein contains a second gene cassette in addition to the first gene cassette or in isolation from the first gene cassette. When the virus does not contain the first gene cassette, the gene cassette is described as a “second gene cassette” for convenience. The second gene cassette contains a promoter and a p53 gene.

Any promoter can be used as long as the promoter drives the p53 gene in cancer-associated non-tumor cells. Examples of the promoters include promoters which are induced in connection with, for example, cell stress such as radiations or virus infection, and promoters which are constitutively expressed in cells. As the promoter, for example, Egr1 (early growth response protein 1) promoters, cytomegalovirus (CMV) promoters, telomerase reverse transcriptase promoters, SV40 late promoters, MMTV LTR promoters, RSV LTR promoters, SRα promoters and the like can be used. As the Egr1 promoter, one derived from mammals can be used. As the Egr1 promoter, one derived from humans, mice, rats, cattle or the like can be used. The sequence of each promoter is known, and for example, for the mouse Egr1 promoter, not only a nucleotide containing a nucleotide sequence set forth as SEQ ID NO: 6, but also the following nucleotides can be used: (a) a nucleotide which is hybridized under stringent conditions with a nucleotide containing a nucleotide sequence complementary to the nucleotide containing a nucleotide sequence set forth as SEQ ID NO: 6; (b) a nucleotide containing a nucleotide sequence having a sequence identity of 80% or more, 85% or more, 90% or more, 95% or more, 98% or more or 99% or more with the nucleotide sequence set forth as SEQ ID NO: 6; and (c) a nucleotide containing a nucleotide sequence having addition, deletion or substitution of one or several bases in the nucleotide sequence set forth as SEQ ID NO: 6. Preferably, the nucleotides of (a) to (c) have Egr1 promoter activity.

The p53 gene is involved in activation of DNA repair proteins when DNA is damaged, control of cell cycles, induction of apoptosis when DNA is subject to unrepairable damage, and the like. It has been first found by the present inventors that expression using a recombinant virus comprising the p53 gene can give damage to cancer-associated non-tumor cells. For the p53 gene, not only a nucleotide containing a nucleotide sequence set forth as SEQ ID NO: 7, but also the following nucleotides can be used: (a) a nucleotide which is hybridized in a stringent condition with a nucleotide containing a nucleotide sequence complementary to the nucleotide containing a nucleotide sequence set forth as SEQ ID NO: 7; (b) a nucleotide containing a nucleotide sequence having a sequence identity of 80% or more, 85% or more, 90% or more, 95% or more, 98% or more or 99% or more with the nucleotide sequence set forth as SEQ ID NO: 7; and (c) a nucleotide containing a nucleotide sequence having addition, deletion or substation of one or several bases in the nucleotide sequence set forth as SEQ ID NO: 7. Preferably, the nucleotides of (a) to (c) encode proteins having p53 activity.

Preferably, the second gene cassette further contains a poly A sequence downstream of the promoter and the p53 gene. When the poly A sequence is contained, mRNA can be protected from decomposition in cell cytoplasm, and at least one function selected from transcription termination, nuclear export and translation can be assisted.

### <3. Other Constitutions of Virus>

Herein, the genes contained in the gene cassette can be obtained by a usual genetic engineering method. For example, it is possible to use a nucleic acid synthesis method using a DNA synthesis apparatus, which is commonly used as a genetic engineering method. It is also possible to use a PCR method comprising isolating or synthesizing gene sequences as templates, and then designing a primer specific to each of the genes, and amplifying the gene sequences using a PCR apparatus; or a gene amplification method using a cloning vector. Those skilled in the art can easily carry out the above-described methods in accordance with “Molecular Cloning, A Laboratory Manual 4th ed.” (described above), or the like.

Thereafter, the respective genes obtained in the manner described above can be linked. First, the respective genes are cut by a known restriction enzyme, and DNA fractions of the cut genes are inserted into a known vector in accordance with a known method, and linked. As the known vector, for example, a pIRES vector can be used. The pIRES vector contains the IRES sequence of an encephalomyocarditis virus (ECMV), and is capable of translating two open reading frames (ORF) from one type of mRNA. When the pIRES vector is used, a polynucleotide containing the first gene cassette can be prepared by sequentially inserting necessary genes into the multi-cloning site. For DNA linking, DNA ligase can be used. By expressing an E1 gene necessary for proliferation of the adenovirus under control of the hTERT promoter, the virus can be proliferated specifically to cancer cells. By incorporating the thus-prepared gene cassette in the virus, a recombinant virus can be prepared.

The recombinant virus described herein (e.g. adenovirus) may lack the E1 region of the virus itself. Since the adenovirus E1 region is involved in replication of the virus, deletion of such a region enables prevention of replication of an undesired virus. In one embodiment, the recombinant adenovirus described herein lacks the E1 region, and contains the first gene cassette at the corresponding position. The E1 region may be deleted in its entirety, or may be partially deleted so as to eliminate its functions.

The recombinant adenovirus described herein may lack the E3 region of the virus itself. In the adenovirus E3 region, ADP (adenovirus death protein) of 11.6 kDa is present. ADP has a function of promoting cytotoxicity and viral spread, and is not a sequence essential to growth of the adenovirus. By deleting the E3 region, the genome size of the recombinant virus can be decreased to introduce a gene with a larger size. In one embodiment, the recombinant adenovirus described herein lacks the E3 region, and contains the second gene cassette at the corresponding position. The E3 region may be deleted in its entirety, or may be partially deleted so as to eliminate its functions.

The type of the virus described herein is not limited, and examples thereof include adenoviruses, adeno associated viruses (AAVs), herpesviruses, retroviruses, lentiviruses, vaccinia viruses, reoviruses, poxviruses and picornaviruses.

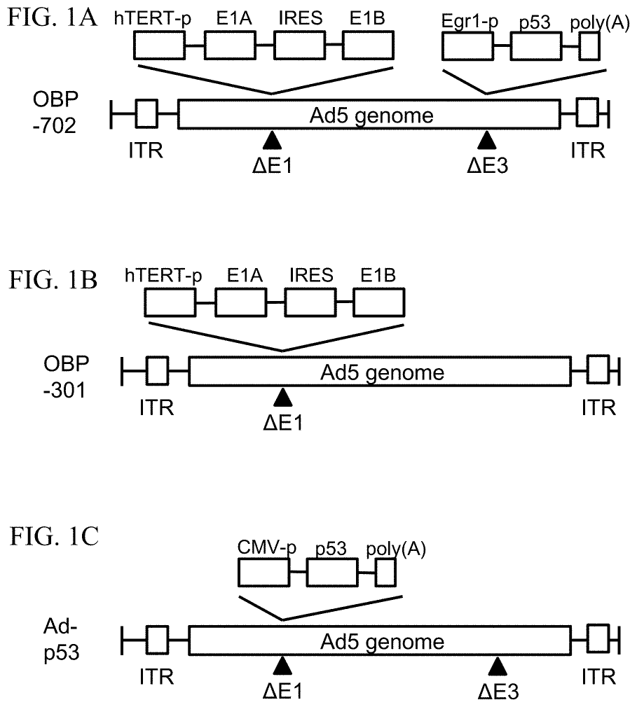

The virus described herein can be easily prepared by those skilled in the art. For example, a virus comprising the gene of FIG. 1 can be easily prepared by replacing the E1 region and the E3 region of the type 5 adenovirus, respectively, with the genes described in FIG. 1 (E1: hTERT-p-E1A-IRES-E1B and E3: Egr1-p-p53-polyA) by using of a kit for gene recombination such as In-fusion.

In one embodiment, the virus described herein treats and/or prevents cancer by damaging cancer-associated non-tumor cells. In one embodiment, the virus described herein may be used for at treating and/or preventing cancer by damaging cancer-associated non-tumor cells.

Since tumor microenvironments constituting a tumor is also related to drug resistance or the like, the virus described herein can also be used for enhancing the effect of other anticancer agents in cancer etc. having drug resistance. The other anticancer agents are not limited, and examples thereof include alkylation agents, platinum preparations, topoisomerase inhibitors, metabolic antagonists, microtubule inhibitors, antibiotic anticancer agents, molecularly targeted agents (including low-molecular compounds and antibody drugs), cell therapies, oncolytic viruses and immunotherapies (including immune checkpoint inhibitors).

Herein, the type of cancer is not limited, and the virus described herein is effective for solid cancers in, for example, stomach, pancreas, large intestine, lung, liver, prostate gland, esophagus, bladder, gallbladder/biliary duct, breast, uterus, thyroid gland and ovary. In particular, pancreas cancer, lung cancer, stomach cancer, esophagus cancer and large intestine cancer are preferable because the tumor contains a large amount of CAF.

Herein, the “cancer-associated non-tumor cells” include tumor-associated fibroblasts (cancer-associated fibroblasts, CAFs), immune cells (e.g. tumor-associated macrophages, T-cells and neutrophils) and mesenchymal stem cells, and CAFs are preferable.

In one embodiment, the virus described herein is an oncolytic virus. In the oncolytic virus, the presence of cancer-associated non-tumor cells may inhibit spread of the virus from one cancer cell to another. Therefore, in one embodiment, since the virus described herein damages cancer-associated non-tumor cells, it has excellent virus spread inhibitory properties and/or an excellent antitumor action.

### <4. Pharmaceutical Composition and Method>

In one embodiment, the present invention relates to a pharmaceutical composition comprising the anti-cancer-associated non-tumor cell preparation agent (also referred to as the “damaging agent described herein” hereinbelow). The pharmaceutical composition of the present invention may contain, in addition to the damaging agent described herein, one or more ingredients selected from known pharmaceutically acceptable carriers such as excipients, extenders, binders and lubricants, and known additives (e.g. buffers, isotonic agents, chelating agents, colorants, preservatives, fragrances, flavors and sweeteners).

In one embodiment, the pharmaceutical composition described herein is used together with other anticancer agents. In one embodiment, the pharmaceutical composition described herein further comprises other anticancer agents. The other anticancer agent is as described above.

In one embodiment, the pharmaceutical composition described herein is used for treating and/or preventing cancer. The type of cancer is as described above. In one embodiment, the pharmaceutical composition described herein is used for treating and/or preventing cancer by damaging cancer-associated non-tumor cells.

The route of administration of the damaging agent or the pharmaceutical composition described herein is not limited, and may be either oral administration or parenteral administration, and the damaging agent or the pharmaceutical composition can be administered to a living body (target cells and organs) by, for example, intravenous, intramuscular, intraperitoneal, intratumor or subcutaneous injection; inhalation through a nasal cavity, an oral cavity or a lung; or a suppository, an external agent or the like.

The form of the damaging agent or the pharmaceutical composition described herein is not limited, and may be a tablet, a capsule, a powder, a granule, a pill, a solution, a syrup, an injection, an external agent, a suppository or an eyedrop.

The dose of the damaging agent or the pharmaceutical composition described herein is appropriately selected according to the type of an active ingredient, the route of administration, the administration target, and the age, the body weight, the sex and the symptom of a patient, or other conditions, and the daily dose can normally be about 10⁶ to 10¹³ PFU, for example about 10⁹ to 10¹² PFU, in terms of the amount of the virus of the present invention which is an active ingredient. The frequency of administration is not limited, and administration can be performed once a day, or in several divided doses.

Examples of the subject to which the damaging agent or the pharmaceutical composition described herein is administered include mammals, for example primates such as humans, laboratory animals such as rats, mice and sewer rats, and livestock animals such as pigs, cattle, horses and sheep, and preferably humans.

In one embodiment, the present invention relates to a method for damaging cancer-related non-tumor cells or a method for treating and/or preventing cancer, the method comprising administrating the virus, the damaging agent or the pharmaceutical composition described herein to a subject. The virus, the damaging agent or the pharmaceutical composition described herein may be administered in a therapeutically effective amount to a cancer-bearing subject.

### EXAMPLES

### Example 1: Preparation of CAF Model

### (Materials and Method)

As cells, fibroblasts (FEF3: human embryonic esophageal fibroblasts (obtained from Wistar Institute) or GFs (gastric fibroblasts): fibroblasts isolated from a stomach tissue (obtained from clinical samples)) were used. GFs were obtained by a method in which a tissue of 6 to 7 mm square is finely minced and cultured (for details, see Tsuyoshi Hasegawa et al., International Journal of Cancer, 134, pp. 1785 to 1795, 2014). FEF3 or GFs were seeded in a 10 cm² dish at a density of 50000 cells/ml, and cultured in DMEM medium under the conditions of 37° C. and 5% CO₂ for 12 to 24 hours. Thereafter, the medium was replaced by DMEM medium containing TGF-β at a concentration of 10 ng/ml, or a cancer-conditioned medium (CM) collected after culturing cancer cells. For CM, a culture supernatant obtained by seeding MKN7 cells in a 175T flask at a density of 28.5×10⁴ cells/mL, culturing the cells in serum-free DMEM medium under the conditions of 37° C. and 5% CO₂ for 72 hours, and then removing cell components by centrifugation was used. 72 to 96 hours after the cells were seeded, expression of αSMA or FAP as a marker of the CAF (cancer-associated fibroblast) in the cells was confirmed by western blotting. That is, first, 20 μg of protein was isolated by SDS-PAGE. Subsequently, the proteins were detected using an anti-αSMA antibody (CST) or an anti-FAP antibody (abcam) as a primary antibody.

### (Results)

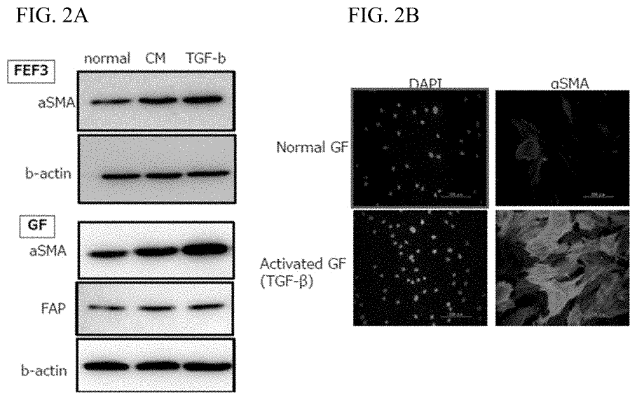

FIG. 2A shows the results of western blotting, and FIG. 2B shows the results of immunostaining. As shown in FIG. 2A, the expression of αSMA and FAP was increased by applying CM or TGF-β to FEF3 and GF which are fibroblasts. As shown in FIG. 2B, the expression of αSMA was increased by applying TGF-β to GF.

These results show that a cell can become CAF by applying CM or TGF-β to the fibroblast.

### Example 2: Damaging Effect of OBP-702 on CAF and Normal Fibroblast

### (Materials and Method)

### Preparation of OBP-702

The density of OBP-702 (obtained from Oncolys BioPharma Inc.) was adjusted with DMEM medium. The sequence of a growth cassette (hTERTp-E1A-IRES-E1B (including a spacer)) contained in OBP-702 is set forth as SEQ ID NO: 8, and the full-length sequence of OBP-702 is set forth as SEQ ID NO: 9.

### XTT Assay

Normal fibroblasts (GFs) (obtained from a clinical sample) and CAF model cells (TGF-β-activated GFs) (prepared by stimulating normal fibroblasts with TGF-β through the method described in Example 1) were seeded in a 96-well plate at a density of 10000 cells/mL, and cultured in DMEM medium under the conditions of 37° C. and 5% CO₂ for 24 hours. One day later, OBP-702 was added at a density of 1 to 100 MOI, or three days later, paclitaxel (obtained from Nippon Kayaku Co. Ltd.) was added at a concentration of 5 to 250 nM. 4 days after the cells were seeded, the damaging rate was determined by an XTT assay using SpectraMax i3.

### (Results)

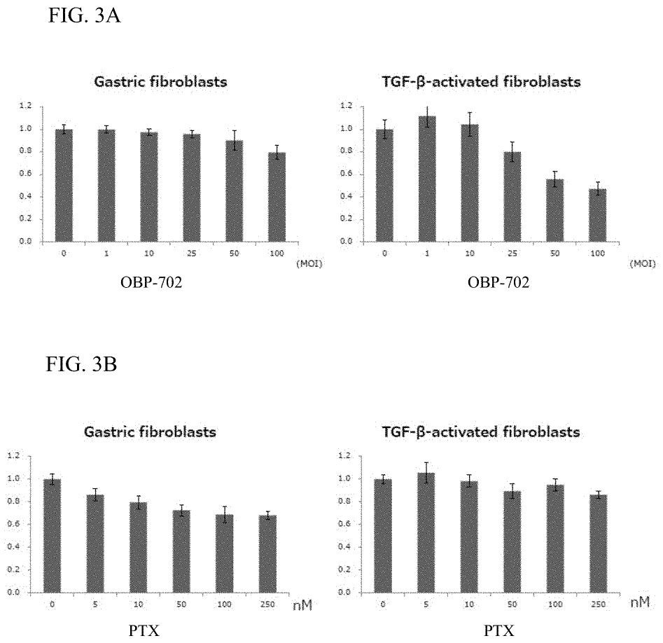

FIG. 3A shows the results for OBP-702, and FIG. 3B shows the results for paclitaxel (PTX). As shown in FIG. 3A, OBP-702 tended to give slight damage to the normal cells and damage CAF model cells in a dose-dependent manner. By contrast, as shown in FIG. 3B, PTX gave slight damage to the CAF model, and damaged the normal cells.

These results show that as compared to PTX, OBP-702 can more effectively damage CAF while suppressing the damage to the normal cells.

### Example 3: Effect of OBP-702 on Expression of Various Proteins in CAF Model Cells

### (Materials and Method)

Fibroblast cells (obtained from a clinical sample) were seeded in a 10 cm² dish at a density of 50000 cells/mL, and cultured in DMEM medium under the conditions of 37° C. and 5% CO₂ for 24 hours. One day later, TGF-β was applied at a concentration of 10 ng/ml. OBP-702 was added at a density of 0 to 100 MOI 4 days later. 4 days after the administration of OBP-702, expression of various proteins was examined by western blotting as follows. That is, first, 20 μg of protein was isolated by SDS-PAGE. Subsequently, the proteins were detected using an anti-αSMA antibody (CST), an anti-FAP antibody (abcam), an anti-p53 antibody (CST), an anti-Ela antibody (BD Pharmingen), an anti-PARP antibody (CST) and an anti-p62 antibody (CST) as primary antibodies. ELISA of IL-6 was carried out using IL-6 ELISA Kit (R&D Systems).

### (Results)

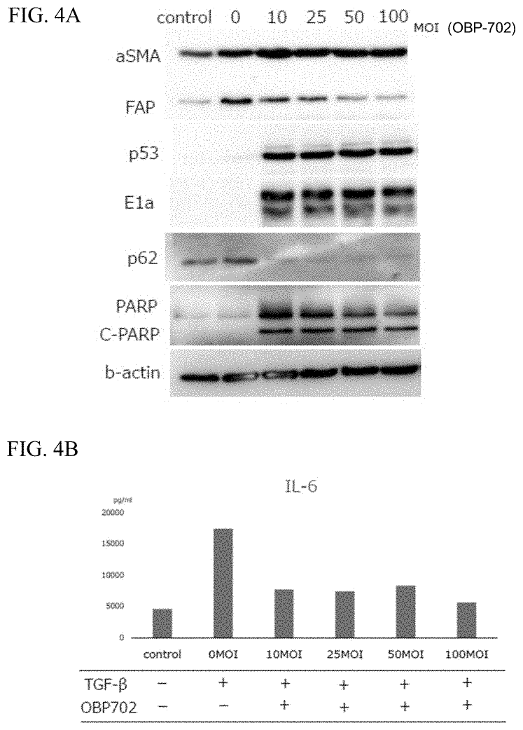

FIGS. 4A and 4B show the results. As shown in FIG. 4A, administration of OBP-702 increased expression of p53 and E1a, and this shows that OBP-702 proliferated. In addition, expression of p62 decreased, showing that autophagy was promoted. In addition, expression of PARP and cleaved PARP (C-PARP) increased, showing that apoptosis was progressed. Further, as shown in FIG. 4B, administration of OBP-702 suppressed production of IL-6.

### Example 4: Examination of Damaging Effect Using CAF Clinical Sample and Normal Fibroblast (NF)

### (Materials and Method)

NFs: fibroblasts isolated from a stomach tissue (obtained from a clinical sample) and a CAF clinical sample taken from a stomach cancer patient (obtained by a method in which a tissue of 6 to 7 mm square is finely minced and cultured; for details, see Tsuyoshi Hasegawa et al. (described above)) were seeded in a 96-well plate at a density of 10000 cells/mL, and cultured in DMEM medium under the conditions of 37° C. and 5% CO₂ for 24 hours. 1 day later, OBP-702, TRAD (OBP-301) which is an infectious recombinant adenovirus described in Japanese Patent No. 3867968, PTX (obtained from Nippon Kayaku Co. Ltd.) and Ad-p53 (obtained from Introgen Therapeutics, Inc.) were each added at a predetermined density/concentration, and the damaging rate was determined by the XTT assay using SpectraMax i3.

### (Results)

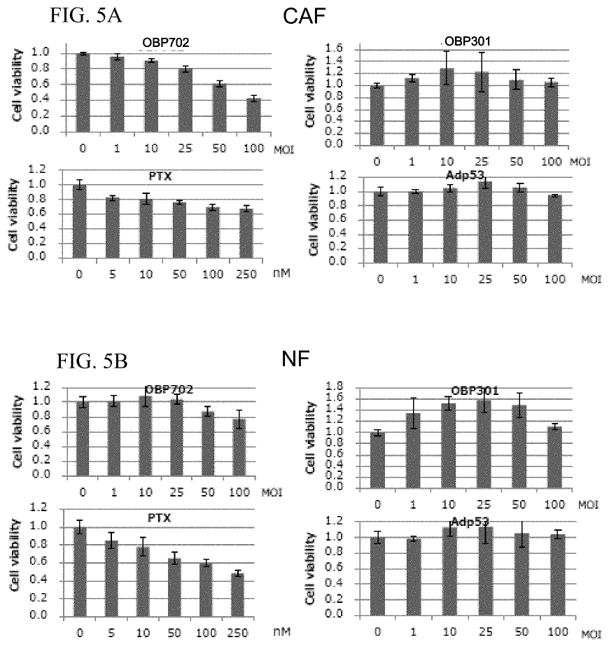

FIG. 5A shows the results for the CAF clinical sample, and FIG. 5B shows the results for NF. OBP-702 gave slight damage to NF and damaged CAF in a dose-dependent manner. OBP-301 comprising the same growth cassette as that of OBP-702 (gene cassette containing hTERT-p, E1A, IRES and E1B) and not comprising a p53 expression cassette, and Ad-p53 comprising a p53 expression cassette and not comprising a growth cassette gave slight damage to NF and CAF. By contrast, PTX damaged CAF, and more heavily damaged NF.

These results show that OBP-702 can more effectively damage CAF while suppressing the damage to normal cells, as compared to other drugs such as PTX.

### Example 5: Damaging Effect on Pancreatic Stellate Cells

### (Materials and Method)

### Preparation of Cancer Conditioned Medium (CM)

Panc-1, MIAPaCa-2, BxPC-3 and Capan-1 were seeded in a 175T flask at a density of 2.0×10⁶ cells/mL, and cultured under the conditions of 37° C. and 5% CO₂ in DMEM medium for Panc-1 and MIAPaCa-2, in RPMI medium for BxPC-3 and in IMDM medium for Capan-1. The medium were replaced by serum-free PSC medium (Thermo Fischer Scientific) at a confluence of 90 to 95% for the cells other than Capan-1 and at a confluence of 60 to 70% for Capan-1. 48 hours later, a culture supernatant was obtained as a cancer-conditioned medium (CM).

### Cell Viability Assay

Fibroblasts (pancreatic stellate cells, hPSC-5 or hPSC-14) were seeded in a 96-well plate at a density of 3000 cells/well (hPSC-5) or 4000 cells/well (hPSC-14), and cultured in PSC medium under the conditions of 37° C. and 5% CO₂. 24 hours later, the medium was replaced by one of four CMs prepared as described above, and 72 hours later, a cell proliferation assay was carried out by the XTT assay using SpectraMax i3. Separately from the cell proliferation assay, 24 hours after the cells were seeded, the medium was replaced by one of four CMs prepared as described above and containing OBP-301 or 702 at a density of 1 to 200 MOI, and 72 hours later, a cell viability assay was carried out by the XTT assay using SpectraMax i3.

### (Results)

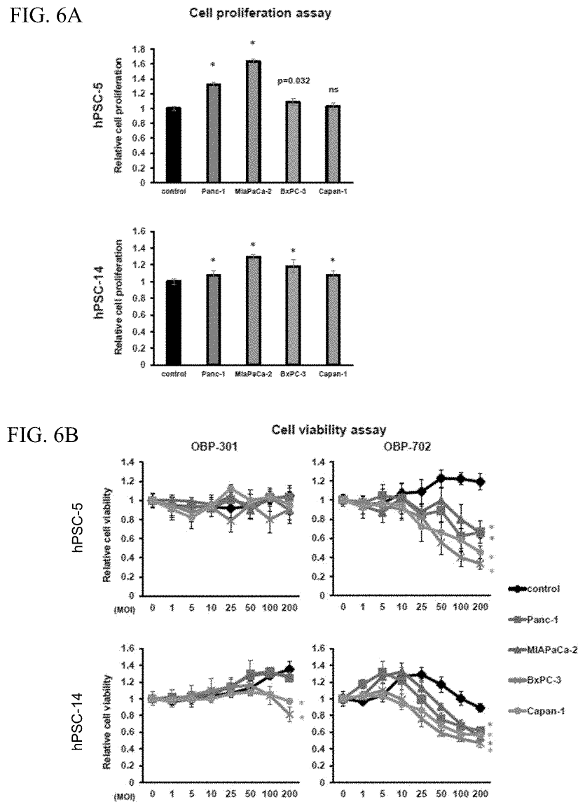

FIG. 6A shows the results of the cell proliferation assay, and FIG. 6B shows the results of the cell viability assay. As shown in FIG. 6A, cells activated with CM more proliferated than the control. As shown in FIG. 6B, administration of OBP-702 gave a significantly more intensified cytotoxicity on the cells to which CM was applied than to the cells to which the control (serum-free medium) was applied.

### Example 6: Change in Expression of Various Proteins in CAF Model Cells by Administration of OBP-702

### (Materials and Method)

Fibroblasts (pancreatic stellate cells, 5×10⁵ cells of hPSC-5 or 7×10⁵ cells of hPSC-14) were cultured in PSC medium under the conditions of 37° C. and 5% CO₂ in a 10 cm dish. 24 hours later, the medium was replaced by Panc-1 CM prepared in Example 5, and OBP-702 was administered at a density of 25 to 100 MOI. 72 hours later, expression of protein was analyzed by western blotting. The western blotting was carried out in the same manner as in Example 3 using an SDS polyacrylamide gel at 10% to 15%.

### (Results)

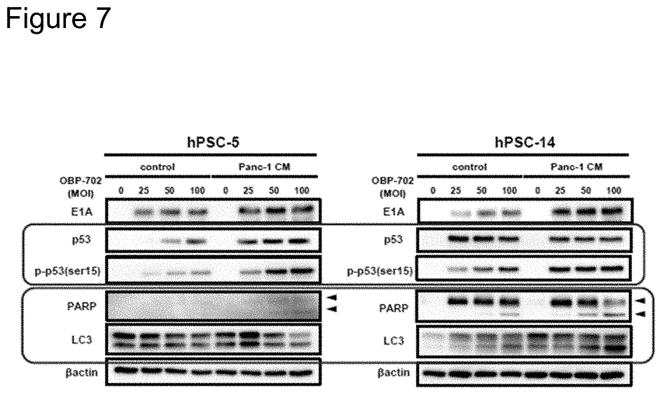

FIG. 7 shows the results. Administration of OBP-702 promoted expression and phosphorylation of p53 and expression of PARP. This indicates that OBP-702 induces cell death through apoptosis and autophagy.

### Example 7: Damaging Effect of TGF-β on CAF Model of Pancreatic Stellate Cells

### (Materials and Method)

Fibroblasts (pancreatic stellate cells, hPSC-5 or hPSC-14) were seeded in a 96-well plate at a density of 3000 cells/well (hPSC-5) or 4000 cells/well (hPSC-14), and cultured in PSC medium under the conditions of 37° C. and 5% CO₂. 24 hours later, TGF-β was applied at 2.5 to 10 ng/mL, and 72 hours later, a cell proliferation assay was carried out by the XTT assay using SpectraMax i3. Separately from the cell proliferation assay, 24 hours after the cells were seeded, TGF-β at 2.5 to 10 ng/mL and OBP-301 or 702 at 1 to 200 MOI were applied, and 72 hours later, a cell viability assay was carried out by the XTT assay using SpectraMax i3.

### (Results)

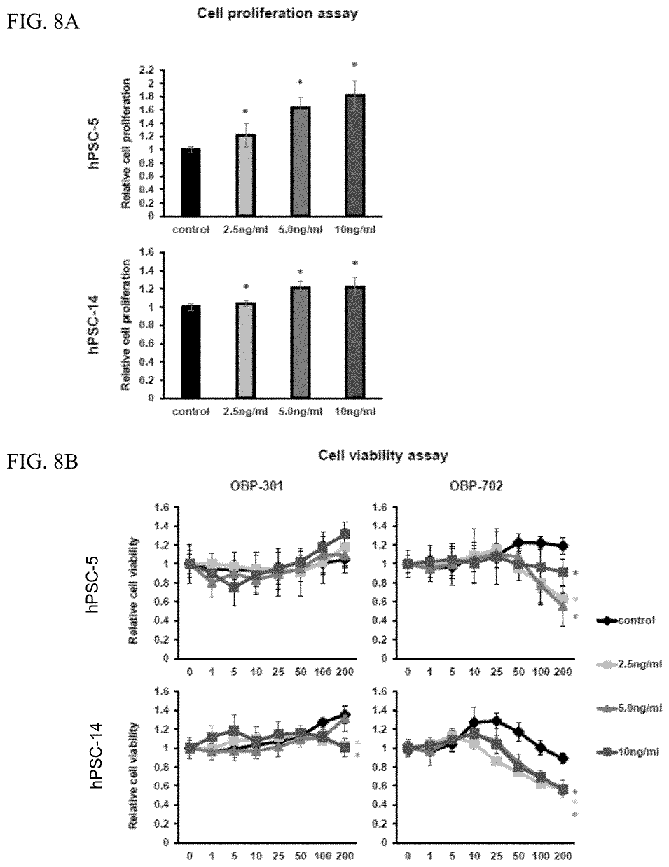

FIG. 8A shows the results of the cell proliferation assay, and FIG. 8B shows the results of the cell viability assay. As shown in FIG. 8A, cells activated with TGF-β more proliferated than the control. As shown in FIG. 8B, administration of OBP-702 gave a significantly more intensified cytotoxicity on cells to which CM was applied than to the cells to which the control (serum-free medium) was applied.

### Example 8: CAF Damaging Effect of AdCMVp53

### (Materials and Method)

Fibroblasts (pancreatic stellate cells, hPSC-5 or hPSC-14) were seeded in a 96-well plate at a density of 3000 cells/well (hPSC-5) or 4000 cells/well (hPSC-14), and cultured in PSC medium under the conditions of 37° C. and 5% CO₂. 24 hours after the cell seeding, the medium was replaced by any of the 4 CMs prepared in Example 5 containing AdCMVp53 (identical to Ad-p53 described in Example 4) or dDL312 which is a controlled growth type 5 adenovirus vector lacking an EIA region (see Patricia C Ryan et al., 2004, Cancer Gene Therapy, volume 11, pages 555-569, and Young C S H et al, 1984, The adenoviruses., pp 125-172) at 1 to 2000 MOI, and 72 hours later, a cell viability assay was carried out by the XTT assay using SpectraMax i3. Separately from four CMs prepared in Example 5, 24 hours after the cells were seeded, TGF-β at 2.5 ng/mL and AdDL312 or AdCMVp53 at 1 to 2000 MOI were applied, and 72 hours later, a cell viability assay was carried out by the XTT assay using SpectraMax i3.

### (Results)

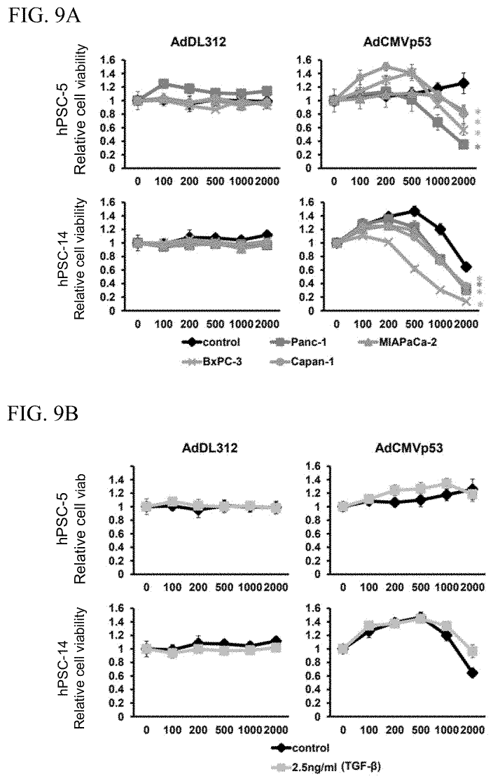

FIG. 9A shows the results for CAF activated with four CMs, and FIG. 9B shows the results for CAF activated with TGF-β. AdDL312 had no damaging effect on the cells to which the control (serum-free medium) was applied or the cells activated with four CMs, whereas AdCMVp53 gave a significantly more intensified damaging effect on cells activated with CM than on cells to which the control (serum-free medium) was applied. These results show that p53 gave a damaging effect on CAF activated with CM. By contrast, neither AdDL312 nor AdCMVp53 had any cytotoxic effect on CAF activated with TGF-β.

### Example 9: Involvement of TGF-β Signaling in Expression of p53

### (Materials and Method)

Fibroblasts (pancreatic stellate cells, 5×10⁵ cells of hPSC-5 or 7×10⁵ cells of hPSC-14) were cultured in PSC medium under the conditions of 37° C. and 5% CO₂ in a 10 cm dish. 24 hours later, the medium was changed to PSC medium containing TGF-β at 2.5 ng/ml, and OBP-702 was administered at 25 to 100 MOI. 72 hours later, expression of protein was analyzed by western blotting. The western blotting was carried out in the same manner as in Example 3 using an SDS polyacrylamide gel at 10% to 15%.

Next, fibroblasts (pancreatic stellate cells, 5×10⁵ cells of hPSC-5) were seeded in a 10 cm dish, and cultured in PSC medium under the conditions of 37° C. and 5% CO₂. 24 hours later, the medium was replaced by Panc-1 CM containing OBP-702 at 100 MOI and a TGF-β inhibitor (R&D Systems) at 0.5 to 2.0 μg/ml, and 72 hours later, expression of p53 was compared by western blotting in the same manner as in Example 3. Fibroblasts (pancreatic stellate cells, hPSC-5 or hPSC-14) were seeded in a 96-well plate at a density of 3000 cells/well (hPSC-5) or 4000 cells/well (hPSC-14), and cultured in PSC medium under the conditions of 37° C. and 5% CO₂. 24 hours later, the medium was replaced by Panc-1 CM or BxPC-3 CM containing OBP-702 at 100 MOI and a TGF-β inhibitor at 0.25 to 2.0 μg/ml, and 72 hours later, a cell proliferation assay was carried out by the XTT assay using SpectraMax i3.

### (Results)

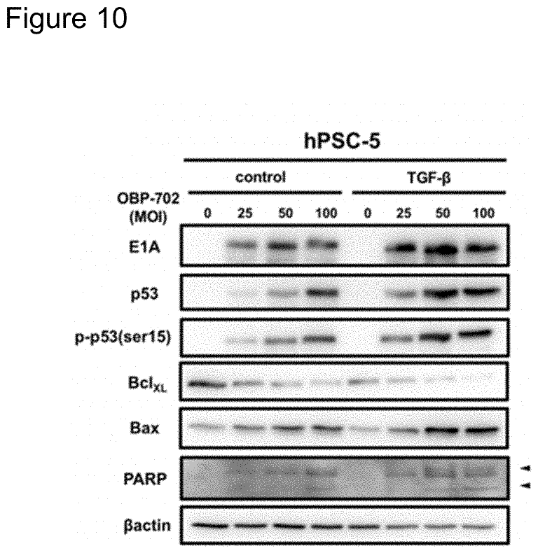

FIG. 10 shows the results. In the TGF-β addition group, the elevation of expression of p53 by OBP-702 increased (FIG. 10). This shows that even in cells which became CAF by TGF-β, OBP-702 increases expression of p53, and enhancement of expression of p53 is related to enhancement of cytotoxic activity by OBP-702 as shown in Example 7.

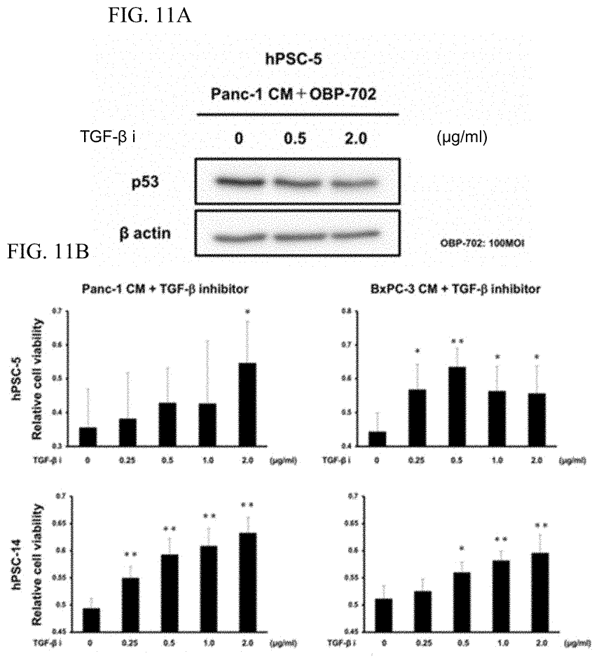

By contrast, when TGF-β contained in Panc-1 CM was inhibited by its inhibitor, the elevation of expression of p53 by OBP-702 decreased, and cytotoxic activity slightly decreased (FIG. 11). This shows that TGF-β contained in the cancer conditioned medium (CM) affects expression of p53 and the CAF damaging effect by OBP-702. In Example 8, AdCMVp53 had no cytotoxicity for CAF activated with TGF-β. This shows that as compared to AdCMVp53, OBP-702 more intensely introduces a p53 gene and exhibits a cytotoxicity for CAF affected by TGF-β which is an important cytokine in tumor microenvironments.

### Example 10: Effect on Tumor Nest in 3D Culture Tissue

### (Materials and Method)

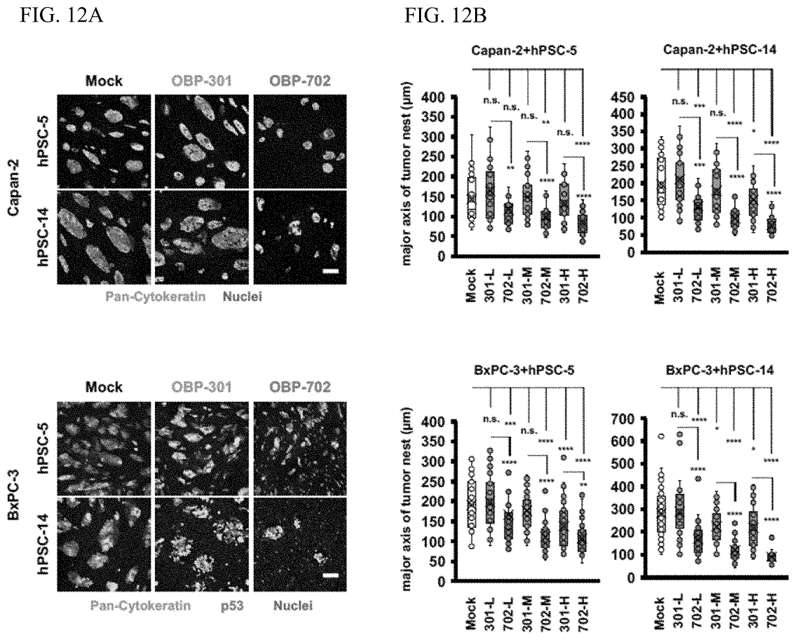

Pancreatic stellate cells (hPSC-5 or hPSC-14) were shaken at room temperature for 30 minutes in a 150 mM isotonic Tris solution containing 0.04 mg/ml fibronectin (Sigma-Aldrich Co. LCC) and 0.04 mg/ml gelatin (FUJIFILM Wako Pure Chemical Corporation) (1 ml in total), and centrifuged (2000 rpm, 2 minutes). 2.5×10⁵ cells of pancreatic stellate cells (hPSC-5 or hPSC-14) coated with fibronectin and gelatin by the above-described method and 5000 cells of Capan-2 were seeded in a culture insert for 24 wells (0.4 μm polyethylene terephthalate transparent membrane, Corning, Inc.), and co-cultured in PSC medium under the conditions of 37° C. and 5% CO₂ to prepare a 3D culture tissue, and the effect of OBP-702 on the 3D culture tissue was evaluated (for details of the 3D culture method, see Hiroyoshi Y Tanaka et al., Biomaterials, 2020, 30; 251: 120077). 96 hours after the cells were seeded, a virus (OBP-301 or OBP-702) was administered. 72 hours after the administration of the virus (120 hours after the cells were seeded), the 3D tissue was immobilized with 4% paraformaldehyde, impregnated with Triton X-100/PBS at 0.2% (v/v), blocked with Blocking One (Nacalai Tesque), and then reacted with a primary antibody at 4° C. overnight. The 3D tissue was washed with PBS, and then reacted with a secondary antibody at 4° C. overnight. Pan-Keratin (C11) Mouse mAb #4545 (CST) and p53 (7F5) Rabbit mAb #2527 (CST) were used as the primary antibody, and Alexa Fluor 488 (anti mouse, Invitrogen Corporation) and Alexa Fluor 568 (anti-rabbit, Invitrogen Corporation) were used as the secondary antibody to perform multi-staining. The 3D tissue was washed with PBS, and then reacted with Hoechst33342 at 4° C. overnight to perform nuclear staining, and the membrane in the culture insert was then cut, mounted on a glass slide, encapsulated with an encapsulating material (Dako/Agilent, Santa Clara), and observed with a confocal laser microscope. The above method enabled evaluation of the size of the tumor nest in the 3D tissue by aggregates of Pan-Keratin-positive cells fluorescently labeled in green, and a change in size of the nest by administration of the virus was evaluated based on the major diameter. The dose of the virus was set to 1.0×10⁶ PFU (Low), 5.0×10⁶ PFU (Middle) and 1.0×10⁷ PFU (High) (in FIG. 12B, 301-L means administration of OBP-301 at Low, 301-M means administration of OBP-301 at Middle, and 301-H means administration of OBP-301 at High. The same applies to OBP-702).

### (Results)

FIG. 12 shows the result. In the 3D culture tissue, OBP-702 reduced the size of the tumor nest more significantly than OBP-301. It is generally known that under co-culture with CAF, the malignancy of pancreas cancer cells increases, and these results show that OBP-702 exhibits high cytotoxic activity through expression of p53 even in cancer cells under co-cultured state. In the model to which OBP-702 was administered, very intensified expression of p53 was visible in a Pan-keratin-negative part (stromal part). These results show that OBP-702 produces expression of p53 even in stromal cell tissues.

### Example 11: Effect on CAF in 3D Culture Tissue

### (Materials and Method)

The 3D models of hPSC5+Capan-2 and hPSC-5+BxPC-3 were prepared in the same process as in Example 10. 96 hours after the cells were seeded, the virus was administered, and 72 hours after the administration of the virus (120 hours after the cells were seeded), the amount of apoptosis cells was evaluated by carrying out a TUNEL (TdT-mediated dUTP Nick End Labeling) assay. Click-iT (trademark) TUNEL Alxa Flour (trademark) 594 Imaging Assay, for microscopy & HCS manufactured by Invitrogen Corporation was obtained, and the TUNEL assay was carried out in accordance with the protocol from Invitrogen Corporation. After TUNEL reaction, multi-staining of the 3D tissue was performed in the same process as in Example 10.

### (Results)

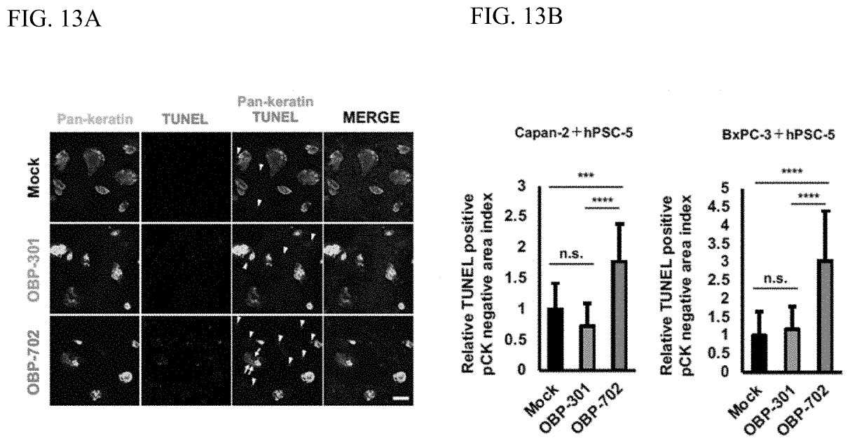

FIG. 13 shows the results. In both the 3D models, quantification of TUNEL expression in the pan cytokeratin-negative part (stromal part, i.e. CAF) showed that OBP-702 significantly increased TUNEL expression as compared to Mock and OBP-301. This shows that OBP-702 induces apoptosis through expression of p53 not only in tumor cells but also in CAF.

### Example 12: Preparation of In Vivo Tumor Cell and Stromal Cell (CAF) Mixed Subcutaneous Tumor Model

### (Materials and Method)

A BxPC-3 mono-injection subcutaneous tumor model obtained by subcutaneously administering 1.0×10⁵ cells of BxPC-3 to a nude mouse (BALB/c-nu/nu) and a BxPC-3+hPSC-14 co-injection subcutaneous tumor model obtained by subcutaneously co-administering 1.0×10⁵ cells of BxPC-3 and 9.0×10⁵ cells of hPSC-14 to a nude mouse (BALB/c-nu/nu) were prepared, and tumor volumes and tumor weights were compared.

### (Results)

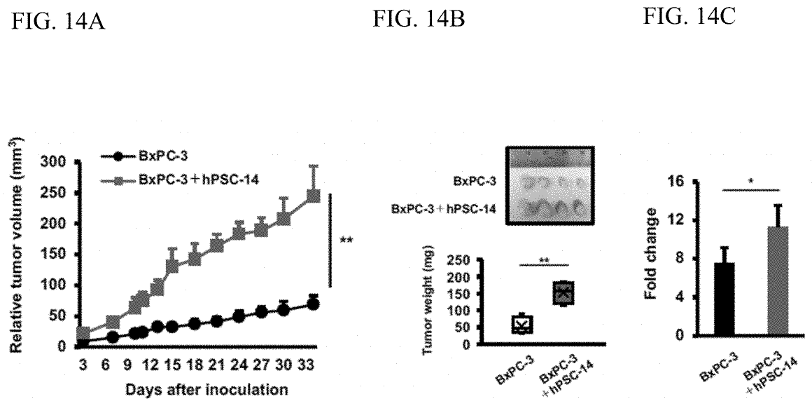

FIG. 14 shows the results. Although the numbers of administered tumor cells were the same, a tumor significant larger in size was formed and the tumor growth rate was significantly higher in the co-injection model, as compared to the mono-injection. This result shows that interaction of PDAC-CAF produced a tumor properties of higher malignancy.

### Example 13: Effect on In Vivo Tumor Cell+Stromal Cell (CAF) Mixed Subcutaneous Tumor Model

### (Materials and Method)

To a BxPC-3+hPSC-14 co-injection model prepared in the same manner as in Example 12, Mock, OBP-301 (1.0×10⁸ PFUs) and OBP-702 (1.0×10⁸ PFUs) were administered three times every other day from 11 days after cell transplantation. The tumor volume was measured, and 22 days after the final administration, the tumor was extracted, and tumor weights were compared.

### (Results)

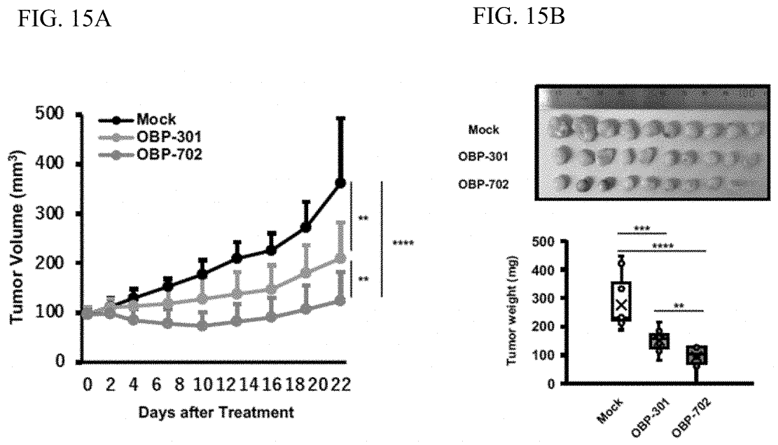

FIG. 15 shows the results. OBP-301 and OBP-702 more significantly suppressed tumor growth as compared to Mock. Further, OBP-702 had a significantly more intense tumor growth suppressing effect as compared to OBP-301. The previous data shows that in the BxPC-3 mono-injection subcutaneous tumor mode, OBP-301 and OBP-702 had equivalent growth suppressing effects (Oncolytic Virus-Mediated Targeting of the ERK Signaling Pathway Inhibits Invasive Propensity in Human Pancreatic Cancer. Mol Ther Oncolytics. 2020 Mar. 31; 17: 107-117). Therefore, the results of this experiment show that in a tumor whose malignancy increases in the presence of a large amount of stroma, OBP-702 exhibits an intense tumor growth suppressing effect while controlling the stroma.

### Example 14: Effect on In Vivo Tumor Cell and Stromal Cell (CAF) Mixed Subcutaneous Tumor Model (2)

### (Materials and Method)

To a BxPC-3+hPSC-14 co-injection model prepared in the same manner as in Example 12, Mock, OBP-301 (1.0×10⁸ PFUs) and OBP-702 (1.0×10⁸ PFUs) were administered every other day from 21 days after cell transplantation. 2 days after the final administration, the tumor was extracted. The extracted tumor was immobilized with paraffin, and pan-cytokeratin and p53 in the tumor tissue were labeled with green fluorescence and red fluorescence, respectively, and observed with a fluorescence microscope. Pan-Keratin (C11) Mouse mAb #4545 (CST) and p53 (7F5) Rabbit mAb #2527 (CST) were used as the primary antibody, and Alexa Fluor 488 (anti mouse, Invitrogen Corporation) and Alexa Fluor 568 (anti rabbit, Invitrogen Corporation) were used as the secondary antibody to perform multi-staining. The reaction time for each of the primary antibody and the secondary antibody was 1 hour.

### (Results)

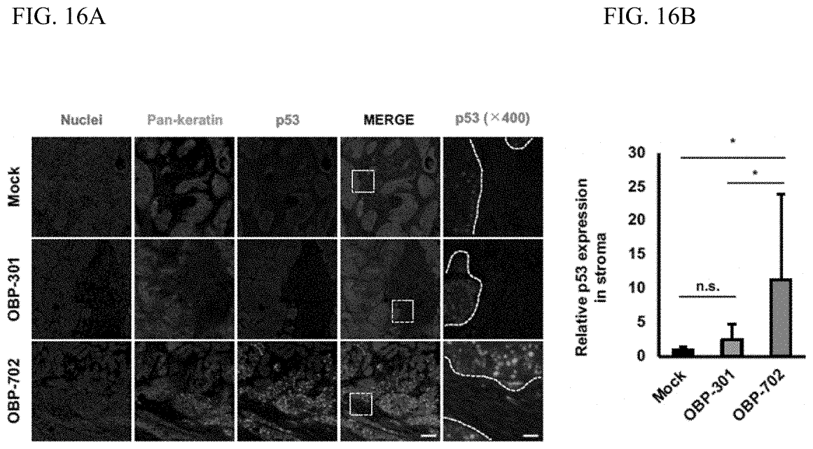

FIG. 16 shows the results. In the OBP-702 administration group, intense expression of p53 locally occurred. The tumor cell part and the stromal part were discriminated from each other by fluorescently labeling Pan-keratin, the intensity of expression of p53 in the stromal part was compared to that of each of the mock group and the OBP-301 administration group. The result showed that in the OBP-702 administration group, expression of p53 in the stroma significantly increased. This result shows that OBP-702 affects not only tumor cells but also stromal cells through expression of p53 even in vivo.

All publications, patents and patent applications cited herein are incorporated herein by reference in their entirety.

### [Sequence Listing]

1. An anti-cancer-associated non-tumor cell agent comprising an oncolytic virus comprising a p53 gene.

2. An anti-cancer-associated non-tumor cell agent comprising a recombinant virus comprising:
a first gene cassette containing a telomerase reverse transcriptase promoter, an E1A gene, an IRES sequence and an E1B gene; and
a second gene cassette containing a promoter and a p53 gene.

3. The anti-cancer-associated non-tumor cell agent according to claim 2, wherein the recombinant virus is an oncolytic virus.

4. The anti-cancer-associated non-tumor cell agent according to claim 2, wherein the first gene cassette contains a telomerase reverse transcriptase promoter, an E1A gene, an IRES sequence and an E1B gene in this order.

5. The anti-cancer-associated non-tumor cell agent according to claim 2, wherein the promoter in the second gene cassette is an Egr1 promoter.

6. The anti-cancer-associated non-tumor cell agent according to claim 1, wherein the cancer-associated non-tumor cell is a cancer-associated fibroblast.

7. The anti-cancer-associated non-tumor cell agent according to claim 1, wherein the virus is a recombinant adenovirus.

8. The anti-cancer-associated non-tumor cell agent according to claim 1, wherein the virus treats and/or prevents a cancer by damaging cancer-associated non-tumor cells.

9. The anti-cancer-associated non-tumor cell agent according to claim 8, wherein the cancer is pancreas cancer or stomach cancer.

10. A pharmaceutical composition comprising the anti-cancer-associated non-tumor cell agent according to claim 1.

11. The anti-cancer-associated non-tumor cell agent according to claim 2, wherein the cancer-associated non-tumor cell is a cancer-associated fibroblast.

12. The anti-cancer-associated non-tumor cell agent according to claim 2, wherein the virus is a recombinant adenovirus.

13. The anti-cancer-associated non-tumor cell agent according to claim 2, wherein the virus treats and/or prevents a cancer by damaging cancer-associated non-tumor cells.

14. The anti-cancer-associated non-tumor cell agent according to claim 13, wherein the cancer is pancreas cancer or stomach cancer.

15. A pharmaceutical composition comprising the anti-cancer-associated non-tumor cell agent according to claim 2.


SEQUENCE LISTING (9 sequences)

SEQ ID NO: 1 - DNA, 455 bp, Homo sapiens
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 2 - DNA, 899 bp, Adenovirus
acaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtg

SEQ ID NO: 3 - DNA, 1823 bp, Adenovirus
ctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatctctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatatgtgggc

SEQ ID NO: 4 - DNA, 620 bp, Picornavirus
tgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgcca

SEQ ID NO: 5 - DNA, 181 bp, Homo sapiens
ccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccct

SEQ ID NO: 6 - DNA, 490 bp, Mus musculus
tgcgccgacccggaaacgccatataaggagcaggaaggatcccccgccggaacagaccttatttgggcagcgccttatatggagtggcccaatatggccctgccgcttccggctctgggaggaggggcgagcgggggttggggcgggggcaagctgggaactccaggcgcctggcccgggaggccactgctgctgttccaatactaggctttccaggagcctgagcgctcgcgatgccggagcgggtcgcagggtggaggtgcccaccactcttggatgggagggcttcacgtcactccgggtcctcccggccggtccttccatattagggcttcctgcttcccatatatggccatgtacgtcacggcggaggcgggcccgtgctgttccagacccttgaaatagaggccgattcggggagtcgcgagagatcccagcgcgcagaacttggggagccgccgccgcgattcgccgccgccgccagcttccg

SEQ ID NO: 7 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 8 - DNA, 3884 bp, Artificial
  Synthetic
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatagatctcgagaattcacgcgaattcggcttacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgaagccgaattcgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgaaattcggcttctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatctctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatatgtgggc

SEQ ID NO: 9 - DNA, 36554 bp, Artificial
  Synthetic
catcatcataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgtaactataacggtcctaaggtagcgaaagctcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattctagcctcgacgcgttggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatagatctcgagaattcacgcgaattcggcttacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgaagccgaattcgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgaaattcggcttctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatctctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatatgtgggcaagccgaatttcgacccgggcggcctagcgtttaaacgggccctctagactcgagcggccgccactgtgctggatgatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcagatctgcagatctgaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaatatgactctcttaaggtagccaaatagggacgaaatcgaagggcccgagctcggtaccgagctcttacgcgtgctagctcgagatctgcgccgacccggaaacgccatataaggagcaggaaggatcccccgccggaacagaccttatttgggcagcgccttatatggagtggcccaatatggccctgccgcttccggctctgggaggaggggcgagcgggggttggggcgggggcaagctgggaactccaggcgcctggcccgggaggccactgctgctgttccaatactaggctttccaggagcctgagcgctcgcgatgccggagcgggtcgcagggtggaggtgcccaccactcttggatgggagggcttcacgtcactccgggtcctcccggccggtccttccatattagggcttcctgcttcccatatatggccatgtacgtcacggcggaggcgggcccgtgctgttccagacccttgaaatagaggccgattcggggagtcgcgagagatcccagcgcgcagaacttggggagccgccgccgcgattcgccgccgccgccagcttccgaagcttggtaccgagctcggatccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacggtcgacctgcaggcatggcggccgcttcgatataacagggtaatattaagttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattatgatgatg